(19) United States
(12) Patent Application Publication
(10) Pub. No.: US 2007/0185200 A1
(43) Pub. Date: Aug. 9, 2007

(54) Screen for pre-eclampsia

(75) Inventors: Makrina Savvidou, London (GB); Aroon Hingorani, London (GB); Patrick Vallance, London (GB); Kypros Nicolaides, London (GB)

Correspondence Address: FULBRIGHT & JAWORSKI L.L.P, 2200 ROSS AVENUE, SUITE 2800, DALLAS, TX, 75201-2784, US

(73) Assignee: University College London, London (GB)

(21) Appl. No.: 10/553,462

(22) PCT Filed: Apr. 19, 2004
(86) PCT No.: PCT/GB04/01701

§ 371 Date: May 5, 2006

(30) Foreign Application Priority Data
Apr. 17, 2003 (GB) 0308967.9

(51) Int. Cl. A61K 31/205; G01N 33/48

(52) U.S. Cl. 514/554; 436/63; 800/3; 800/9

(57) ABSTRACT

It has been demonstrated that the level of asymmetric dimethylarginine (ADMA) increases in women that subsequently develop pre-eclampsia or whose fetus subsequently develops intrauterine growth restriction (IUGR) and that ADMA plays a key role in the development of maternal hypertension. Accordingly, the level of ADMA in a pregnant woman can be used to determine whether or not a pregnant woman is at risk of developing pre-eclampsia or whether or not a fetus is at risk of developing IUGR. Furthermore, antagonists of ADMA activity are useful in the inhibition or prevention of pre-eclampsia or inhibition or prevention of IUGR.

### FIELD OF THE INVENTION

The invention relates to the diagnosis of susceptibility to and the prevention of the development of pre-eclampsia and intrauterine growth restriction (IUGR).

### BACKGROUND OF THE INVENTION

Pre-eclampsia is a common disorder affecting 3 to 5% of human pregnancies and accounting for 40% of iatrogenic deliveries in the UK. It is part of a group of hypertensive disorders that also includes eclampsia, latent chronic essential hypertension, renal diseases and transient gestational hypertension. The disorder is typically defined as acute hypertension accompanied by abnormal proteinuria developing after 20 weeks geststaion in a previously normotensive woman (Davey D A et al. Am J Obstet Gynecol 1988; 158: 892-8). It is also typically associated with pulmonary oedema, cyanosis, impaired liver function, visual or cerebral disturbances, pain in the epigastric area or right upper quadrant, decreased platelet count, intrauterine-growth restriction (IUGR) or oliguria. At present, the pathogenesis of pre-eclampsia is poorly understood, limiting the development of a reliable predictive test or effective prophylaxis.

### SUMMARY OF THE INVENTION

Asymmetric dimethylarginine (ADMA) is an endogenous inhibitor of nitric oxide synthase (NOS) that competes with binding of the natural substrate L-arginine. It is produced from methylated arginine residues in proteins by protein methyltransferases (PRMT) and is metabolised by the enzyme dimethylarginine dimethylaminohydrolase (DDAH). ADMA is produced in the fetoplacental unit, which contains large amounts of DDAH II. The inventors have shown for the first time that levels of ADMA are increased in women that subsequently develop pre-eclampsia and whose children develop IUGR.

According to the invention there is thus provided a method of identifying whether or not a pregnant woman is at risk of developing pre-eclampsia or whether or not her fetus is at risk of developing intrauterine growth restriction (IUGR), which method comprises measuring ADMA in the pregnant woman and thereby determining whether or not the woman is at risk of developing pre-eclampsia or determining whether or not her fetus is at risk of developing IUGR.

The invention further provides:
use of an ADMA antibody for the manufacture of means for determining whether or not a woman is at risk of developing pre-eclampsia or determining whether or not her fetus is as risk of developing IUGR;
a method of inhibiting or preventing pre-eclampsia in a pregnant woman or inhibiting of preventing IUGR in her fetus, comprising administering to the pregnant woman an effective amount of an antagonist of ADMA activity;
use of an antagonist of ADMA activity for the manufacture of a medicament for inhibiting or preventing pre-eclampsia or inhibiting or preventing IUGR;
a non-human pregnant female animal in which pre-eclampsia has been established by administration of ADMA;
a non-human pregnant female animal in which IUGR has been established in her fetus by administration of ADMA;
a non-human fetus in which IUGR has been established by administration of ADMA to a non-human female animal that is pregnant with the fetus;
a method for establishing pre-eclampsia or establishing IUGR in her fetus in a non-human pregnant female animal comprising administering ADMA to the animal in an amount sufficient to cause pre-eclampsia or IUGR,
a method of identifying a substance which prevents or treats pre-eclampsia or prevents or treats IUGR, comprising administering a candidate substance to an animal in which pre-eclampsia or IUGR has been established and assessing whether or not the candidate substance prevents or treats pre-eclampsia or prevents or treats IUGR;
a method of identifying a substance which prevents or treats pre-eclampsia or treats or prevents IUGR, comprising administering a candidate substance to a pregnant DDAH deficient animal and assessing whether or not the candidate substance prevents or treats pre-eclampsia or prevents or treats IUGR; and
use of a substance identified by a method of the invention for the manufacture of a medicament for preventing or treating pre-eclampsia or preventing or treating IUGR

### DESCRIPTION OF THE FIGURES

FIG. 1 shows flow-mediated dilatation (FMD) of the brachial artery at 23 to 25 weeks of gestation at the different group of women depending on the presence or absence of bilateral notches and on the outcome of pregnancy. The horizontal lines illustrate the mean FMD in each group.

FIG. 2 shows plasma levels of ADMA in the two groups of pregnant women (with and without bilateral notches in the uterine artery Doppler examination). The horizontal lines illustrate the median ADMA levels in each group.

FIG. 3 shows scatterplot illustrating the relationship between flow-mediated dilatation and plasma levels of ADMA in the group of pregnant women who had bilateral notches of the uterine arteries at 23 to 25 weeks of gestation and eventually developed pre-eclampsia. A significant inverse correlation was found (r=−0.8, p=0.005).

FIG. 4 shows synthesis of ADMA from methylated arginine residues in proteins by protein methylarginases (PRMT), and its metabolism to citrulline through the action of the dimethylarginine dimethyldiaminohydrolases I and II (DDAH I and II).

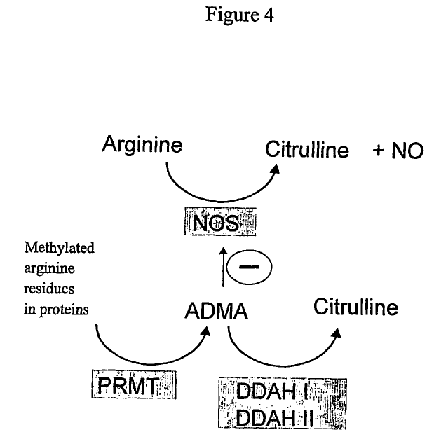

### DETAILED DESCRIPTION OF THE INVENTION

Diagnosis

The present invention relates to a method of identifying whether or not a pregnant woman is at risk of developing pre-eclampsia or whether or not her fetus is at risk of developing IUGR. The invention therefore relates to the diagnosis of susceptibility of a pregnant woman to pre-eclampsia and the diagnosis of susceptibility of a fetus to IUGR. The pregnant woman is a human being. The fetus is human. The woman or fetus who is diagnosed may be in the first, second or third trimester of pregnancy. Typically the woman or fetus is at a stage of pregnancy from 4 to 25 weeks gestation. The woman or fetus may be at a stage of pregnancy from 23 to 25 weeks gestation. Preferably the woman or fetus is at a stage of pregnancy from 10 to 25 weeks gestation and more preferably from 15 to 25 weeks gestation.

Typically the woman does not have pre-eclampsia or displays no symptoms of pre-eclampsia. However, the method of the invention may be carried out on women who have pre-eclampsia but have not been tested for it. The method may therefore be carried out on women who display preliminary symptoms of pre-eclampsia.

Typically the fetus does not have IUGR or displays no symptoms of IUGR. However, the method of the invention may be carried out on women whose fetuses have IUGR but have not been tested for it. The method may therefore be carried out on women whose fetuses display preliminary symptoms of IUGR.

The present invention involves measuring ADMA in the woman. Typically the level or concentration of ADMA is measured. According to the present invention, an increased level of ADMA compared with the normal pregnancy level indicates that the woman is susceptible to or at risk of developing pre-eclampsia or her fetus is susceptible to or at risk of developing IUGR. The normal pregnancy level is typically the level of ADMA in a woman who displays no symptoms of pre-eclampsia or whose fetus does not display symptoms of IUGR throughout the entire pregnancy. The normal pregnancy level is typically at an equivalent stage of pregnancy.

The mean plasma ADMA concentration in a normal non-pregnant population is typically about 0.82 μmol/L. Generally the mean plasma ADMA concentration in a normal pregnant human is lower than that in a normal non-pregnant individual and remains relatively constant throughout pregnancy. The normal pregnancy level for a human at 4, 10, 15 and 25 weeks of pregnancy is typically about 0.3 to 0.6 μmol/L, for example 0.52 μmol/L in plasma.

In the present invention, an increased plasma level of ADMA associated with increased susceptibility to or risk of developing of pre-eclampsia or increased susceptibility to or risk of developing of IUGR is typically greater than about 1.45 μmol/L, greater than about 1.5 μmol/L or greater than about 2.0 μmol/L. According to the present invention, the ADMA level/concentration in the woman is preferably increased by at least 3 fold and typically by at least 4 fold compared to the normal pregnancy level. The ADMA level/concentration is typically raised by 3 to 7 fold compared to the normal pregnancy level. According to the present invention, an ADMA level or concentration above the 95% confidence interval for the normal pregnancy level is typically associated with an increased risk of developing pre-eclampsia or IUGR

Symmetric dimethylarginine (SDMA) is the biologically inactive stereosiomer of ADMA. SDMA is not metabolized by DDAH but is instead excreted by the kidney. The L-arginine/ADMA ratio is typically used as an index of NOS inhibition. The ADMA/SDMA ratio typically reflects DDAH activity. The present invention may also involve assessment of the ADMA/SDMA ratio to determine the risk of developing pre-eclampsia.

According to the present invention, an increased ADMA/SDMA ratio compared with the normal pregnancy ratio indicates that the woman is susceptible to or at risk of developing pre-eclampsia or her fetus is susceptible to or at risk of developing IUGR. The normal pregnancy ratio is the ratio of ADMA/SDMA in a woman who displays no symptoms of pre-eclampsia or whose fetus displays no symptoms of IUGR throughout the entire pregnancy. The normal ADMA/SDMA ratio is typically at an equivalent stage of pregnancy.

The normal pregnancy ratio for a human at 4, 10, 15 and 25 weeks of pregnancy is typically about 1:1 to 1.3:1, for example 1.3:1 in plasma. In the present invention, an increased plasma ratio of ADMA/SDMA associated with increased susceptibility to or risk of developing of pre-eclampsia or IUGR at 23 to 25 weeks is typically about 6.8:1. According to the present invention, the ADMA/SDMA ratio in the woman is preferably increased by at least 5 fold and more preferably by at least 6 fold compared to the normal pregnancy level, for example at the same stage of pregnancy. The ADMA/SDMA ratio is typically increased by 5 to 8 fold compared to the normal pregnancy level. According to the present invention, an ADMA/SDMA ratio above the 95% confidence interval for the normal pregnancy ratio is associated with an increased risk of developing pre-eclampsia or IUGR.

The invention is typically carried out in vitro on a sample obtained from the woman. The sample typically comprises a body fluid of the woman. The sample is preferably a blood, plasma, serum or urine sample but may be amniotic fluid. The sample is typically processed prior to being assayed, for example by centrifugation. The sample may also be typically stored prior to assay, preferably below −70° C.

Standard methods known in the art may be used to assay the level of ADMA. These methods typically involve contacting the sample with an antibody capable of binding to ADMA. Such methods include dipstick assays and Enzyme-linked Immunosorbant Assay (ELISA). Typically dipsticks comprise one or more antibodies or proteins that specifically bind ADMA. If more than one antibody is present, the antibodies preferably have different non-overlapping determinants such that they may bind to ADMA simultaneously.

ELISA is a heterogeneous, solid phase assay that requires the separation of reagents. ELISA is typically carried out using the sandwich technique or the competitive technique. The sandwich technique requires two antibodies. The first specifically binds ADMA and is bound to a solid support. The second antibody is bound to a marker, typically an enzyme conjugate. A substrate for the enzyme is used to quantify the ADMA-antibody complex and hence the amount of ADMA in a sample. The antigen competitive inhibition assay also typically requires an ADMA-specific antibody bound to a support. An ADMA-enzyme conjugate is added to the sample (containing ADMA) to be assayed. Competitive inhibition between the ADMA-enzyme conjugate and unlabeled ADMA allows quantification of the amount of ADMA in a sample. The solid supports for ELISA reactions preferably contain wells.

The present invention may also employ methods of measuring ADMA that do not comprise antibodies. High Performance Liquid Chromatography (HPLC) separation and fluorescence detection is preferably used as a method of determining the ADMA level. HPLC apparatus and methods as described previously may be used (Tsikas D et al. J Chromatogr B Biomed Sci Appl 1998; 705: 174-6) Separation during HPLC is typically carried out on the basis of size or charge. Prior to HPLC, endogenous amino acids and an internal standard L-homoarginine are typically added to assay samples and these are phase extracted on CBA cartridges (Varian, Harbor City, Calif.). Amino acids within the samples are preferably derivatized with o-phthalaldehyde (OPA). The accuracy and precision of the assay is preferably determined within quality control samples for all amino acids.

The present invention may be used to confirm susceptibility in women already suspected as being at risk or selected as being predisposed to developing pre-eclampsia. The present invention may also be used to confirm susceptibility in fetuses already suspected as being at risk or selected as being predisposed to developing IUGR. Risk factors that increase susceptibility to developing pre-eclampsia or IUGR typically include Afro-Caribbean ancestry, null parity or first pregnancy with a partner, multiple gestations, hypertension, diabetes, genetic predisposition to or family history of pre-eclampsia or eclampsia, obesity, hypercholesterolaemia and smoking. The present invention may be used to determine the susceptibility of developing pre-eclampsia in smokers. The present invention may also be used to determine the susceptibility of the fetus of a smoker developing IUGR.

Some embodiments of the invention include additional diagnostic tests to determine susceptibility to pre-eclampsia of IUGR. Flow-mediated dilation of the brachial artery and/or Doppler waveform analysis of the uterine arteries are preferably employed. These diagnostic tests are typically carried out before, at the same time as or after measurement of the ADMA level in a pregnant woman.

Flow-mediated dilatation (FMD) of the brachial artery is an established non-invasive method of assessing endothelium-dependent vasodilation. It typically involves measuring changes in brachial artery diameter in response to increased flow using high resolution ultrasound. Ultrasonic apparatus and methods previously described may be used (Savvidou M D et al. Ultrasound Obstet Gynecol 2000; 15: 502-7; Dorup I et al Am J Physiol 1999; 276: H821-5). The apparatus typically includes a linear array transducer. End-diastolic images of the artery may be stored in digital format. Arterial diameter is typically determined using a semi-automated edge detection algorithm. Baseline vessel diameter is typically calculated as the mean of all the measurements during the first minute of recording. FMD of the brachial artery is defined as the percentage increase in vessel diameter during reactive hyperaemia induced by inflation of a cuff distal to the site of the recording to 300 mm Hg for 5 minutes followed by rapid deflation. Flow change (reactive hyperemia), an index of the flow stimulus for dilation, is typically calculated as [(blood flow 15 sec after cuff deflation-baseline blood flow)/baseline blood flow]×100%. Endothelium-independent dilatation to sublingual glyceryl trinitrate (GTN) may be used as a control.

FMD of the brachial artery is typically 8.59±2.76% (n=43) in normal pregnant human women. According to the invention, a significant reduction of FMD in addition to an increased AMDA level is indicative of a susceptibility to or a risk of developing pre-eclampsia. The FMD of the brachial artery is preferably reduced by two fold or more preferably by at least two fold.

Doppler analysis is a routine method used to assess the waveform of the uterine arteries. The method is typically used to identify the presence or absence of early diastolic notches in the artery waveform. These notches may be unilateral or bilateral. According to the invention, the presence of a unilateral notch or bilateral notches in addition to an increased AMDA level is indicative of a susceptibility to pre-eclampsia or IUGR.

The diagnostic method of the invention may be carried out in conjunction with other assays or genetic tests to refine risk prediction.

The invention further provides a diagnostic kit that comprises means for measuring the ADMA level in a woman and thereby determining whether or not the woman is at risk of developing pre-eclampsia or her fetus is susceptible to IUGR. The kit typically contains one or more antibodies that specifically bind ADMA. For example, the kit may comprise a monoclonal antibody, a polyclonal antibody, a single chain antibody, a chimeric antibody, a CDR-grafted antibody or a humanized antibody. The antibody may be an intact immunoglobulin molecule or a fragment thereof such as a Fab, F(ab′)₂ or Fv fragment. If more than one antibody is present, the antibodies preferably have different non-overlapping determinants such that they may bind to ADMA simultaneously.

The kit may additionally comprise one or more other reagents or instruments which enable any of the embodiments of the method mentioned above to be carried out. Such reagents or instruments include one or more of the following: suitable buffer(s) (aqueous solutions), means to isolate ADMA from sample, means to obtain a sample from the woman (such as a vessel or an instrument comprising a needle) or a support comprising wells on which quantitative reactions can be done. The kit may, optionally, comprise instructions to enable the kit to be used in the method of the invention or details regarding which women the method may be carried out upon. The kit may, optionally, comprise an antagonist of ADMA activity. The antagonist is preferably L-arginine.

Therapy

The present invention also relates to the inhibition or prevention of pre-eclampsia and the inhibition or prevention of IUGR. The inventors have shown that ADMA levels are increased in women that subsequently develop pre-eclampsia and that ADMA plays a key role in the development of maternal hypertension by attenuating endothelium-dependent relaxation. The inventors have also shown that ADMA levels are increased in pregnant women whose fetuses subsequently develop IUGR. The development of pre-eclampsia or IUGR may therefore be prevented or inhibited by using antagonists of AMDA activity.

The inhibition of pre-eclampsia involves reducing, preventing or delaying the symptoms of pre-eclampsia in a pregnant woman who already has pre-eclampsia. The prevention of pre-eclampsia involves reducing, preventing or delaying pre-eclampsia in a pregnant woman who does not have pre-eclampsia but is at risk of developing the condition.

The inhibition of IUGR involves reducing, preventing or delaying the symptoms of IUGR in a fetus that already has IUGR. The prevention of IUGR involves reducing, preventing or delaying IUGR in a fetus that does not have IUGR but is at risk of developing the condition. The conditions of fetuses at risk of developing IUGR or displaying the symptoms of IUGR can therefore be improved by administration of a substance used in the inhibition or prevention of IUGR. A therapeutically effective amount of a substance used in the inhibition or prevention of the development of IUGR is preferably given to the mother of the fetus.

Another aspect of the present invention is the treatment of a pregnant woman identified by a method of the invention as at risk of developing pre-eclampsia. Thus, a substance used in the inhibition or prevention pre-eclampsia may be used in the manufacture of a medicament for use in the treatment of pregnant woman identified by a method of the invention as at risk of developing pre-eclampsia. The conditions of pregnant woman identified by a method of the invention as at risk of developing pre-eclampsia can therefore be improved by administration of a substance used in the inhibition or prevention pre-eclampsia. A therapeutically effective amount of a substance used in the inhibition or prevention of the development of pre-eclampsia may be given to a woman identified by a method of the invention as in need thereof.

Another aspect of the present invention is the treatment of a fetuses identified by a method of the invention as at risk of developing IUGR. Thus, a substance used in the inhibition or prevention IUGR may be used in the manufacture of a medicament for use in the treatment of fetuses identified by a method of the invention as at risk of developing IUGR. The conditions of fetuses identified by a method of the invention as at risk of developing IUGR can therefore be improved by administration of a substance used in the inhibition or prevention of IUGR. A therapeutically effective amount of a substance used in the inhibition or prevention of the development of IUGR is preferably given to the mother of the fetus identified by a method of the invention as in need thereof.

The woman who has been identified as at risk of developing pre-eclampsia and therefore undergoing treatment may be in the first, second or third trimester of pregnancy. The fetus who has been identified as at risk of developing IUGR and therefore undergoing treatment may be in the first, second or third trimester of pregnancy. Typically the woman or fetus is at a stage of pregnancy from 4 to 25 weeks gestation. The woman or fetus may be at a stage of pregnancy from 23 to 25 weeks gestation. Preferably the woman or fetus is at a stage of pregnancy from 10 to 25 weeks gestation and more preferably from 15 to 25 weeks gestation.

Fully developed pre-eclampsia is typically treated by delivery of the fetus. Development of pre-eclampsia is preferably prevented using antagonists of ADMA activity. Development of IUGR is also preferably prevented using antagonists of ADMA activity. Antagonists of AMDA activity typically reduce the concentration or level of AMDA and/or inhibit its effects. The antagonist of AMDA activity is preferably L-arginine, which is the natural substrate for nitric oxide synthase and competes with ADMA. The antagonist of ADMA activity may also be an inhibitor of PRMT or a stimulator of DDAH.

Inhibition or prevention of pre-eclampsia also typically involves anti-hypertensive therapy. Inhibition or prevention of IUGR may also involve anti-hypertensive therapy. Hypertensive therapy may be pharmacological or non-pharmacological. Preferred non-pharmacological methods of prevention include hospitalization, stopping smoking and/or continuous monitoring of blood pressure, protein levels, platelet count, renal function and other standard indicators of cardiovascular function. Preferred pharmacological methods of prevention/and or management include administration of magnesium sulphate, hydralazine, labetalol or aspirin to the woman.

In the invention, substances used in the inhibition or prevention of pre-eclampsia or IUGR may be administered in a variety of dosage forms. Thus, they can be administered orally, for example as tablets, troches, lozenges, aqueous or oily suspensions, dispersible powders or granules. They may also be administered parenterally, either subcutaneously, intravenously, intramuscularly, intrasternally, transdermally or by infusion techniques. They may also be administered as suppositories. A physician will be able to determine the required route of administration for each particular patient.

The formulation of a substance used in the inhibition or prevention of pre-eclampsia or IUGR according to the invention will depend upon factors such as the nature of the exact antagonist, etc. A suitable substance may be formulated for simultaneous, separate or sequential use.

A substance used in the inhibition or prevention pre-eclampsia or IUGR according to the invention is typically formulated for administration in the present invention with a pharmaceutically acceptable carrier or diluent. The pharmaceutical carrier or diluent may be, for example, an isotonic solution. For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

A therapeutically effective amount of a substance used in the inhibition or prevention of pre-eclampsia or IUGR is administered to a patient identified according to a method of the invention. The dose, for example of an ADMA antagonist, may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific antagonist, the age, weight and conditions of the subject to be treated and the frequency and route of administration. Preferably, daily dosage levels are from 5 mg to 2 g. That dose may be provided as a single dose or may be provided as multiple doses, for example taken at regular intervals, for example 2, 3 or 4 doses administered daily.

Animal Model

The present invention also provides an animal in which pre-eclampsia has been established and a method of generating such an animal. The inventors have shown that ADMA plays a key role in the development of pre-eclampsia. ADMA may therefore be used to generate an animal that displays symptoms similar to those displayed by a pregnant women who has been diagnosed with pre-eclampsia. The animal of the invention is suitable for use as a model for studying pre-eclampsia.

The present invention also provides a pregnant animal in which IUGR has been established in her fetus and a method of generating such an animal. The inventors have shown that ADMA plays a key role in the development of IUGR. ADMA may therefore be used to generate an animal whose fetus displays symptoms similar to those displayed by a human fetus who has been diagnosed with IUGR. The animal of the invention is suitable for use as a model for studying IUGR.

The present invention also provides an animal fetus in which IUGR has been established and a method of generating such an animal. The inventors have shown that ADMA plays a key role in the development of IUGR. ADMA may therefore be used to generate a fetus that displays symptoms similar to those displayed by a human fetus who has been diagnosed with IUGR. The animal of the invention is suitable for use as a model for studying IUGR.

ADMA is administered in a sufficient amount to cause or generate pre-eclampsia symptoms in the animal or to cause or generate IUGR symptoms in the fetus. The sufficient amount typically varies between animals and will depend on a number of factors, for example plasma volume and normal pregnancy level of ADMA.

ADMA may be administered to the animals by methods well known in the art. ADMA can be administered orally, for example as tablets, troches, lozenges, aqueous or oily suspensions, dispersible powders or granules. ADMA may also be administered parenterally, either subcutaneously, intravenously, intramuscularly, intrasternally, transdermally or by infusion techniques.

The animal is non-human. The non-human animal is typically of a species commonly used in biomedical research, for example a mammal, and is preferably a laboratory strain. Suitable animals include non-human primates, dogs, cats, sheep and rodents. It is preferred that the animal is a rodent, particularly a mouse, rat, guinea pig, ferret, gerbil or hamster. Most preferably the animal is a mouse.

The animal may also lack functional dimethylarginine dimethylaminohydrolase (DDAH), the enzyme which metabolises ADMA. DDAH deficient animals have been described previously in WO 00/44888 (PCT/GB00/00226). A DDAH deficient animal is not capable of expressing an active form of DDAHI and/or a DDAH II. An animal which is not capable of expressing one or more isoforms of DDAH is one which shows substantially no detectable expression of at least one DDAH mRNA. An animal which is not capable of expressing an active form of one or more isoforms of DDAH is one which expresses at least one DDAH related polypeptide, which polypeptide shows substantially no DDAH activity.

A suitable animal may be one in which the polynucleotide sequence from a DDAH encoding gene locus has been deleted or replaced with polynucleotide sequences from another locus or from another organism. Thus, substantially no DDAH mRNA may be expressed from that DDAH locus. Alternatively, the coding sequence of a DDAH gene may have been altered such that the expressed polypeptide shows substantially no DDAH activity.

Typically a suitable non-human animal is a so-called “knock-out animal”. The term “knock-out animal” is well known to those skilled in the art. Typically, a non-human animal of the invention, for example a knock-out animal, will be a transgenic animal.

A knock-out animal can be produced according to any suitable method. In general, a polynucleotide construct is produced comprising a marker gene, for example, flanked by genomic sequences. Those genomic sequences correspond to genomic sequences at the DDAH encoding gene locus of the animal in question. Thus, if the polynucleotide construct is contacted with the DDAH encoding gene locus of the animal of interest, homologous recombination events may lead to replacement of the chromosomal sequence bordered by the genomic sequences used in the polynucleotide construct with the marker gene. If the marker gene replaces coding sequence or a regulatory sequence, for example a promoter sequence, gene expression and/or activity may be abolished.

The polynucleotide construct is typically transferred into a fertilized egg by pronuclear microinjection so that the contacting described above can occur. Alternative approaches may be used for example, embryonic stem cells or retroviral mediated gene transfer into germ lines. Whichever approach is taken, transgenic animals are then generated. For example, microinjected eggs may be implanted into a host female and the progeny may be screened for the expression of the marker gene. The founder animals that are obtained may be bred.

Preferred animals are thus mice in which all or part of the DDAHI or DDAHII gene locus has been deleted or replaced for example, i.e. DDAHI or DDAHII knock-out mice.

The animal may also over-express an enzyme involved in the endogenous synthesis or activity of ADMA, for example PRMT. The transgenic technology described above is of course equally applicable to the production of non-human animals which over-express a protein. In such cases the polynucleotide construct used does not replace an endogenous portion of a gene with a marker gene. Instead, an endogenous gene may be replaced with a polynucleotide construct comprising a promoter, for example one which drives high levels of expression, operably lined to a coding sequence. Alternatively, the construct may comprise an appropriate promoter sequence operably linked to a reporter gene. It is also possible to produce constructs which do not replace endogenous sequences. Use of such constructs will result in animals which contain endogenous sequences and the sequences insert by the construct.

The transgenic non-human animals described above may also be used independently for the identification of substances that prevent or treat pre-eclampsia or IUGR described below.

The animal is pregnant. The term pregnant is herein defined as a condition wherein the animal behaves physiologically and responds to pharmacological treatment as if it were pregnant. The invention may therefore employ animals in pregnancy-like states such as, for example, pseudopregnancy.

Screening for Therapeutic Substances

The present invention further provides a method of using the whole or part of an animal to identify substances that prevent or treat pre-eclampsia or IUGR. This method typically uses the non-human pregnant animal of the invention or the non-human fetus of the invention. However, in one embodiment, a pregnant DDAH deficient animal, as described above, is used without prior ADMA administration. The lack of DDAH, the enzyme which metabolises ADMA, causes an elevation in the endogenous level of ADMA. The DDAH deficient animal is preferably a knock out mouse.

Substances which prevent pre-eclampsia reduce, prevent or delay the appearance of any symptoms of pre-eclampsia. Substances which treat pre-eclampsia alleviate or abolish the symptoms of pre-eclampsia in an individual who has been diagnosed with the condition.

Substances which prevent IUGR reduce, prevent or delay the appearance of any symptoms of IUGR. Substances which treat IUGR alleviate or abolish the symptoms of IUGR in an fetus that has been diagnosed with the condition.

The method of identifying substances is typically carried out before or after the symptoms of pre-eclampsia have developed during the pregnancy of the animal. The method of identifying substances that prevent pre-eclampsia is typically carried out before the symptoms of pre-eclampsia have developed in the animal. The method of identifying substances that treat pre-eclampsia are typically carried out after the symptoms of pre-eclampsia have developed in the animal.

The method of identifying substances is typically carried out before or after the symptoms of IUGR have developed in the fetus. The method of identifying substances that prevent IUGR is typically carried out before the symptoms of IUGR have developed in the fetus. The method of identifying substances that treat IUGR are typically carried out after the symptoms of IUGR have developed in the animal.

Suitable substances which can be tested in the above method include combinatorial libraries, defined chemical entities, peptide and peptide mimetics, oligonucleotides and natural product libraries, such as display (e.g. phage display libraries) and antibody products. For example, monoclonal and polyclonal antibodies, single chain antibodies, chimeric antibodies, CDR-grafted antibodies and humanized antibodies may be used. The antibody may be an intact immunoglobulin molecule or a fragment thereof such as a Fab, F(ab′)₂ or Fv fragment.

Typically, organic molecules will be screened, preferably small organic molecules which have a molecular weight of from 50 to 2500 daltons. Candidate products can be biomolecules including saccharides, fatty acids, steroids, purines, pyrimidines, derivatives, structural analogs or combinations thereof. Candidate agents are obtained from a wide variety of sources including libraries of synthetic or natural substances. Known pharmacological agents may be subjected to directed or random chemical modifications, such as acylation, alkylation, esterification, amidification, etc. to produce structural analogs.

Preferred test substances include substances that affect the level or activity of ADMA, NOS, NO, DDAH or PRMT.

The invention also provides for use of the substances identified by the screening method of the invention in the prevention or treatment of pre-eclampsia or IUGR. Accordingly, the identified substances may be used in the manufacture of a medicament for use in the prevention or treatment of pre-eclampsia or IUGR

The following Example illustrates the invention:

### EXAMPLE

1. Methods

Study Participants

All women attending for routine antenatal care at King's College Hospital had colour Doppler examination of their uterine arteries at 23-25 weeks of gestation (Acuson Aspen, Calif., USA). The Doppler waveforms of the uterine arteries were obtained as previously described (Albaiges G et al. Obstet Gynecol 2000; 96: 559-64). When three similar consecutive waveforms were acquired the presence of an early diastolic notch was noted, and the mean pulsatility index (PI) of the two vessels was calculated. 43 pregnant women with abnormal uterine artery Doppler waveforms presence of early diastolic notch bilaterally) identified consecutively, were recruited at the time of the Doppler study, and matched for age, ethnic group and smoking status with 43 pregnant women with normal uterine artery Doppler waveforms. At entry, all women had singleton pregnancies, were healthy, on no medications, had no personal or family history of premature cardiovascular disease, and had appropriately grown fetuses for the gestation. Maternal age, ethnic group, smoking status, parity, heart rate and BP were recorded. BP was measured in the right arm with the subject seated using an ambulatory blood pressure monitor (SpaceLabs Medical 90207, WA, USA). Three measurements were taken and averaged. The study was approved by the Local Ethics Committee and all subjects gave written informed consent

Assessment of Maternal Endothelial Function

Ultrasound of the right brachial artery was performed using a 7 MHz linear array transducer and an Aspen Acuson system (California, USA) as previously described, at 23-25 weeks' gestation (Celermajer D S et al. Lancet 1992; 340: 1111-5; Savvidou M D et al. Obstet Gynecol 2000; 15: 502-7) End-diastolic images of the artery were acquired every 3 seconds and stored in digital format. Arterial diameter was determined for each image using a semi-automated edge detection algorithm. Baseline vessel diameter was calculated as the mean of all the measurements during the first minute of recording. FMD of the brachial artery was defined as the percentage increase in vessel diameter during reactive hyperaemia induced by inflation of a cuff distal to the site of the recording to 300 mm Hg for 5 minutes followed by rapid deflation. Flow change (reactive hyperemia), an index of the flow stimulus for dilation, was calculated as [(blood flow 15 sec after cuff deflation-baseline blood flow)/baseline blood flow]×100%. All the measurements were performed by an experienced operator and the FMD data analysed within 24 h of the study. In our laboratory the interobserver variability for FMD is 1.02±0.6% (95% limits of agreement: −1.7-2.4%) (Savvidou M D et al. Obstet Gynecol 2000; 15: 502-7). Outside the setting of pregnancy, endothelium-independent dilatation to sublingual glyceryl trinitrate (GTN) is commonly used as a control but in the current study GTN use was avoided. However, a previous study has shown that GTN-induced dilatation is not altered as a result of pregnancy (Dorup I et al. Am J Physiol 1999; 276: H821-5).

Analysis of Plasma Asymmetric-, Symmetric-Dimethylarginines and L-Arginine

A blood sample (5 ml) was taken into citrate tubes at the time of the vascular studies for the measurement of plasma concentrations of ADMA, SDMA and L-arginine. Thirty eight women from the control group and 40 women with bilateral uterine artery notches agreed to blood sampling. After centrifugation the plasma was stored at −70° C. until assay. Endogenous amino acids and the internal standard L-homoarginine added to 0.5 ml aliquots of plasma samples (at 10 μmol/L) were solid-phase extracted on CBA cartridges (Varian, Harbor City, Calif.), derivatized with o-phthalaldehyde (OPA), and the OPA derivatives were separated by HPLC and monitored by fluorescence detection as described (Tsikas D et al. J Chromatogr B Biomed Sci Appl 1998; 705: 174-6). OPA derivatives of L-arginine, L-homoarginine, SDMA and ADMA eluted at 8.8±0.1, 10.9±0.1, 14.8±0.2, and 16.3±0.2 min, respectively, (mean±SD, n=6). The accuracy and precision were determined within a set of six co-processed quality control samples to be closely to 100% and below 7.3%, respectively, for all amino acids.

Definition of Clinical Outcome

Information on the course of the pregnancy, including gestational age, mode of delivery and infant birth weight was obtained for all the women studied. The clinical management of the women participating in the study was undertaken by obstetricians who were aware of the results of the uterine artery waveform recordings at 23-25 weeks, but were unaware of the results of brachial artery FMD and methylarginine measurements. Pre-eclampsia was defined according to the criteria of the International Society for the Study of Hypertension in Pregnancy (Davey et al. Am J Obstet Gynecol 1988; 158: 892-8). Under this classification, pre-eclampsia was defined as hypertension (one diastolic blood pressure reading≧110 mm Hg, or two consecutive diastolic blood pressure readings≧90 mm Hg at least four hours apart) in combination with proteinuria (≧300 mg total protein in a 24-hour urine collection or, if this was not available, 2+ proteinuria by dipstick on two consecutive occasions at least four hours apart) developing after 20 weeks of gestation in previously normotensive women. IUGR was defined as birth weight below the 5^{th} percentile for gestation and sex of the neonate (Gardosi J et al. Lancet 1992; 339: 283-7).

Statistical Analysis

Normality of the distribution of continuous data was examined with the Shapiro-Wilk test. Logarithmic transformation was performed for non-normally-distributed data. Descriptive data are expressed as mean±SD or as median [interquartile range] for normally and non-normally distributed data. The Student's t-test was used to compare variables between the women with and without bilateral notches. Comparisons between multiple groups were performed using one-way analysis of variance followed by a post hoc test (Tukey-Kramer). Chi-square (χ²) test was used to compare categorical variables among groups. Univariate linear and multivariate regression analyses were performed where appropriate. The statistical analyses were performed using the Statistical Package for Social Sciences (Version 8).

2. Results

Baseline Characteristics of the Study Participants

None of the women with normal uterine artery Doppler waveforms developed pre-eclampsia and all of them delivered infants of appropriate size. Women with bilateral notches of the uterine arteries at 23-25 weeks (n=43) were classified into three groups according to the outcome of pregnancy; those with no complications (n=19, 44.2%), those who developed IUGR (n=14, 32.6%) and those who developed PE (n=10, 23.2%), including four with IUGR.

The demographic and clinical characteristics, obtained at study entry, according to the outcome of the pregnancy are presented in Table 1. There was no statistically significant difference between the groups in baseline demographic characteristics. A greater proportion of women who subsequently developed pre-eclampsia were smokers, but the difference was not statistically significant (p=0.43). Systolic and diastolic BP were significantly higher in women who eventually developed pre-eclampsia but were nevertheless within the normal range. Women who developed pre-eclampsia had significantly higher PI of the uterine arteries and delivered smaller fetuses earlier compared to the women who did not have any complications of pregnancy.

Maternal FMD According to the Outcome of Pregnancy

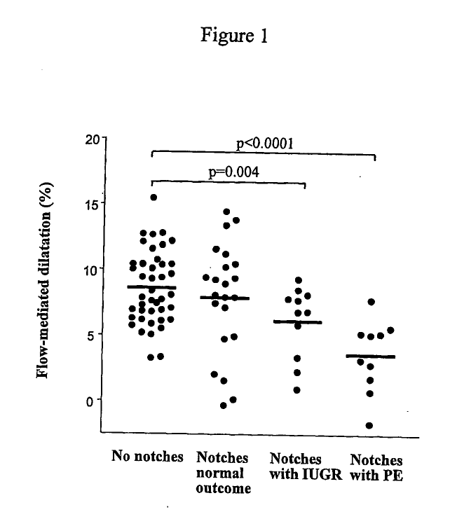

Recordings of FMD were obtained from all women and are presented in Table 2 according to the outcome of pregnancy. Women who subsequently developed pre-eclampsia, had significantly lower FMD (3.58±2.76%) than women who had no notches and normal outcome (8.59±2.76%, p<0.0001) and women who had bilateral notches but a normal outcome (8.15±4.32%, p=0.0001, FIG. 1). Women whose pregnancies were complicated by IUGR also had lower FMD compared to women who had no notches and normal outcome (6.17±2.82% vs. 8.59±2.76%, p=0.004). Pregnant women with normal outcome had similar FMD regardless of the absence or presence of bilateral notches (8.59±2.76% vs. 8.15±4.32%, p=0.92). In a multiple regression analysis, significant predictors of FMD were baseline vessel size (p=0.002), subgroup status defined by pregnancy outcome (p<0.001) and smoking status (p<0.001). The difference in FMD among the groups remained significant even after adjustment for smoking status.

Levels of Dimethylarginines and L-Arginine

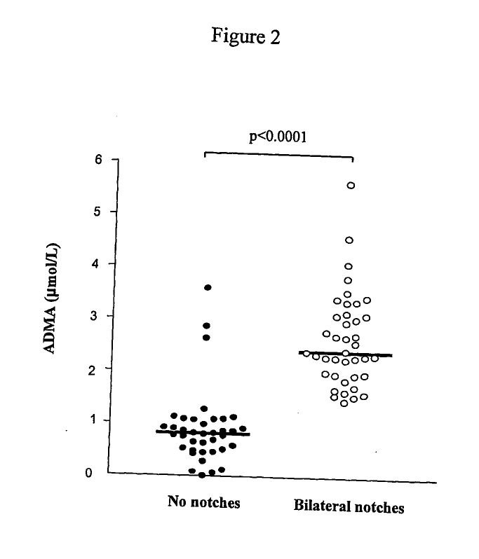

Women with bilateral notches had significantly higher levels of ADMA compared to the women with normal uterine artery Doppler waveforms (2.4 [1.97-3.14] μmol/L vs. 0.81 [0.49-1.08] μmol/L respectively, p<0.0001, FIG. 2). Women who subsequently developed pre-eclampsia had significantly higher levels of ADMA compared to the women who had normal pregnancies (2.7 [2.21-3.21] μmol/L vs. 0.81 [0.49-1.08] μmol/L respectively, p<0.0001, Table 3). At the levels seen in women who subsequently develop pre-eclampsia, ADMA competitively inhibits the enzymatic synthesis of NO from L-arginine and attenuates endothelium-dependent relaxation. ADMA provides a mechanism for the development of pre-eclampsia and links increased placental vascular resistance with maternal hypertension resulting from systemic maternal endothelial dysfunction. The levels of symmetric dimethylarginine (SDMA; a stereoisomer of ADMA with no effect on NO synthesis) were similar in all groups, with the result that the ADMA/SDMA molar ratio was significantly higher in women with bilateral notches and pre-eclampsia (Table 3). L-arginine concentration and L-arginine/ADMA molar ratio were marginally but significantly higher in all women with bilateral notches and pre-eclampsia when compared to those without. Interestingly, despite having the lowest FMD, women who went on to develop PE had the highest rather than the lowest levels of L-arginine (Table 3).

Correlation of ADMA with Endothelial Function

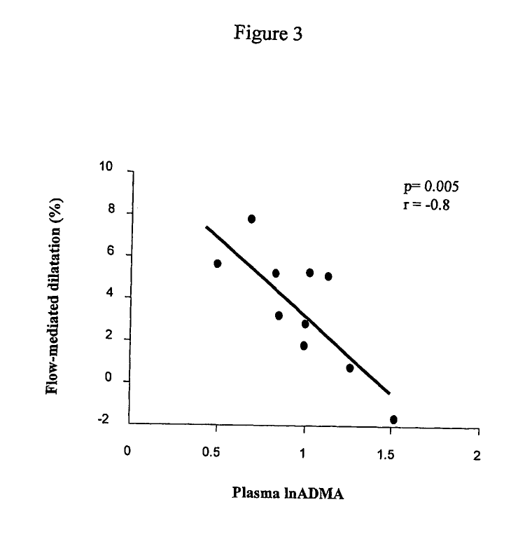

The levels of ADMA did not correlate significantly with FMD in the group of women with normal Doppler waveforms. However, there was a weak but significant inverse correlation between the plasma levels of ADMA and FMD in the group of women with bilateral uterine artery notches (r=−0.35, p=0.02). In order to investigate this relationship further, we performed univariate analyses between levels of ADMA and FMD in the three subgroups of women with bilateral notches distinguished by different pregnancy outcomes. Only in the group of pregnant women who eventually developed pre-eclampsia, there was a strong inverse correlation between ADMA and FMD (r=−0.8, p=0.005, FIG. 3).

TABLE 1
Demographic, clinical maternal and neonatal characteristics in
each group of women, according to the outcome of pregnancy.

| | No | Notches | Notches | Notches |
|---|---|---|---|---|
| | notches- | present- | present- | present- |
| | Normal | Normal | Development | Development |
| | outcome | outcome | of IUGR | of PE |
| Characteristic | (n = 43) | (n = 19) | (n = 14) | (n = 10) |
| Gestational age at | 23 (23-25) | 24 (24-25) | 24 (24-25) | 24 (24-25) |
| screening (wk) | | | | |
| Maternal age | 29 ± 5.3 | 27.1 ± 5.8 | 26.3 ± 6.2 | 28.2 ± 4.7 |
| (years) | | | | |
| Smokers | 7 (16.3%) | 2 (10.5%) | 4 (28.6%) | 3 (30%) |
| Nulliparity | 22 (51.2%) | 10 (52.6%) | 12 (85.7%) | 7 (70%) |
| Heart rate (bpm) | 83.1 ± 10.1 | 77.5 ± 13.1 | 80.2 ± 10.6 | 79.5 ± 11.4 |
| Systolic BP | 113.9 ± 8.9 | 114.2 ± 7.2 | 114.7 ± 9.6 | 125.6 ± 7.2* |
| (mmHg) | | | | |
| Diastolic BP | 66.7 ± 6.8 | 64.2 ± 16 | 66 ± 5.9 | 75.4 ± 9† |
| (mmHg) | | | | |
| Mean PI of uterine | 0.81 ± 0.23 | 1.49 ± 0.6† | 1.85 ± 0.7* | 1.69 ± 0.5† |
| arteries | | | | |
| Gestational age at | 39.4 ± 1.6 | 40 ± 1.7 | 38.6 ± 4 | 34.8 ± 3.5* |
| delivery (wk) | | | | |
| Birth weight (gr) | 3326 ± 460 | 3207 ± 408 | 2301 ± 727* | 2074 ± 723* |

*p < 0.0001,
†p < 0.001
All the comparisons were performed with the group of women without bilateral notches at examination.

TABLE 2
Maternal vascular characteristics in each group
of women, according to the outcome of pregnancy.

| | No | Notches | Notches | Notches |
|---|---|---|---|---|
| | notches- | present- | present- | present- |
| | Normal | Normal | Development | Development |
| | outcome | outcome | of IUGR | of PE |
| Characteristic | (n = 43) | (n = 19) | (n = 14) | (n = 10) |
| Flow-mediated | 8.59 ± 2.76 | 8.15 ± 4.32 | 6.17 ± 2.82* | 3.58 ± 2.76†‡ |
| dilatation, FMD | | | | |
| (%) | | | | |
| Baseline vessel | 3.1 ± 0.35 | 3.17 ± 0.38 | 3.3 ± 0.52 | 3.2 ± 0.4 |
| diameter (mm) | | | | |
| Baseline blood | 182.2 | 181.5 | 128.6 | 194.4 |
| flow (ml/min) | (90.3-278) | (82.5-210.6) | (86-319) | (100-367) |
| Reactive hyperemia | 473 | 614.5 | 605.5 | 381.3 |
| (%) | (245-805) | (359-925) | (215-926) | (163-940) |

*p = 0.004, comparison with women without notches and normal outcome.
†p < 0.0001, comparison with women without notches and normal outcome.
‡p < 0.0001, comparison with women with notches and normal outcome.

TABLE 3
Levels of ADMA, SDMA and L-arginine in each group
of women, according to the outcome of pregnancy.

| | No | Notches | Notches | Notches |
|---|---|---|---|---|
| | notches- | present- | present- | present- |
| | Normal | Normal | Development | Development |
| | outcome | outcome | of IUGR | of PE |
| Characteristic | (n = 38) | (n = 16) | (n = 14) | (n = 10) |
| Plasma ADMA | 0.81 | 1.99* | 3.04* | 2.78 |
| (μmol/L) | (0.49-1.08) | (1.69-2.38) | (2.39-3.54) | (2.21-3.21) |
| Plasma SDMA | 0.61 | 0.51 | 0.54 | 0.51 |
| (μmol/L) | (0.43-0.76) | (0.18-0.69) | (0.34-0.7) | (0.27-0.77) |
| Plasma L-arginine | 21.9 | 23.5 | 28.3† | 31.1* |
| (μmol/L) | (19.4-25.8) | (22.2-27.6) | (23.6-33.7) | (24.6-35) |
| ADMA/SDMA | 1.27 | 4.6* | 5.65* | 6.8* |
| molar ratio | (0.97-1.68) | (3.1-13.5) | (3.97-8.87) | (3.92-8.72) |
| L-arginine/ADMA | 31.1 | 11.09* | 8.93* | 11.27* |
| molar ratio | (21.4-39.7) | (8.85-15) | (7.36-14.17) | (8.74-15.17) |

*p < 0.0001,
†p < 0.005
All the comparisons were performed with the group of women without bilateral notches at examination.

1. A method of identifying whether or not a pregnant woman is at risk of developing pre-eclampsia or whether or not her fetus is at risk of developing intrauterine growth restriction (IUGR), which method comprises measuring asymmetric dimethylarginine (ADMA) in the pregnant woman and thereby determining whether or not the woman is at risk of developing pre-eclampsia or determining whether or not her fetus is at risk of developing IUGR.

2. A method according to claim 1, wherein ADMA is measured in a fluid sample taken from the woman.

3. A method according to claim 1, wherein determining whether or not the woman is at risk of developing pre-eclampsia or determining whether or not her fetus is at risk of developing IUGR comprises determining whether or not the ADMA is greater than 1.5 μmol/L in the fluid sample.

4. A method according to claim 1, wherein the pregnant woman is at a stage of pregnancy from 10 to 25 weeks gestation.

5. A method according to claim 4, wherein the woman is at a stage of pregnancy from 15 to 25 weeks gestation.

6. A method according to claim 1, wherein determining whether or not the woman is at risk of developing pre-eclampsia or determining whether or not her fetus is at risk of developing IUGR comprises determining whether or not the woman's ADMA level is at least 3 times the normal pregnancy level.

7. A method according to claim 1, wherein determining whether or not the woman is at risk of developing pre-eclampsia or determining whether or not her fetus is at risk of developing IUGR comprises determining whether or not the woman has an increase in the ADMA/symmetric dimethylarginine (ADMA/SDMA) ratio that is greater than the normal pregnancy ratio.

8. A method according to claim 7, comprising determining whether or not the ADMA/SDMA ratio is at least 5 times more than the normal pregnancy ratio.

9. A method according to claim 1, wherein the pregnant woman is suspected of being at risk of developing pre-eclampsia or her fetus is suspected of being at risk of developing IUGR.

10. A method according to claim 9, wherein the woman is a smoker.

11. A method according to claim 1, further comprising carrying out Doppler waveform analysis of the uterine arteries and/or flow-mediated dilatation of the brachial artery in the woman.

12. (canceled)

13. (canceled)

14. (canceled)

15. (canceled)

16. (canceled)

17. (canceled)

18. An animal which is:
(a) a non-human pregnant female animal in which pre-eclampsia has been established by administration of ADMA;
(b) a non-human pregnant female animal in which IUGR has been established in her fetus by administration of ADMA; or
(c) a non-human fetus in which IUGR has been established by administration of ADMA to a non-human female animal that is pregnant with the fetus.

19. (canceled)

20. (canceled)

21. A method which is:
a) a method for establishing pre-eclampsia in a non-human pregnant female animal or establishing IUGR in her fetus comprising administering ADMA to the animal in an amount sufficient to cause pre-eclampsia or IUGR;
(b) a method of identifying a substance which prevents or treats pre-eclampsia or prevents or treats IUGR, comprising administering a candidate substance to an animal as defined in claim 18 and assessing whether or not the candidate substance prevents or treats pre-eclampsia or prevents or treats IUGR;
(c) a method of identifying a substance which prevents or treats pre-eclampsia or prevents or treats IUGR, comprising administering a candidate substance to a pregnant DDAH deficient animal and assessing whether or not the candidate substance prevents or treats pre-eclampsia or prevents or treats IUGR; or
(d) a method of inhibiting or preventing pre-eclampsia in a pregnant
woman or inhibiting or preventing IUGR in her fetus, comprising administering to the pregnant woman an effective amount of an antagonist of ADMA activity.

22. A non-human pregnant female animal according to claim 18, a non-human fetus according to claim 18 or a method according to claim 21, wherein the non-human pregnant female animal is a dimethylarginine dimethylaminohydrolase (DDAH) deficient animal.

23. (canceled)

24. (canceled)

25. The method according to claim 21, wherein the DDAH deficient animal is a knockout mouse.

26. (canceled)

27. A method according to claim 21, wherein the woman has been identified as at risk of developing pre-eclampsia or her fetus has been identified as at risk of developing IUGR by a method according to claim 1.

28. A method according to claim 21, wherein the antagonist of ADMA activity is L-arginine.
